(19) United States
(12) Patent Application Publication
(10) Pub. No.: US 2004/0058886 A1
(43) Pub. Date: Mar. 25, 2004

(54) Short interfering RNAs having a hairpin structure containing a non-nucleotide loop

(75) Inventor: Stephen Scaringe, Lafayette, CO (US)

Correspondence Address: KALOW & SPRINGUT LLP, 488 MADISON AVENUE, 19TH FLOOR, NEW YORK, NY, 10022, US

(73) Assignee: Dharmacon, Inc., Lafayette, CO

(21) Appl. No.: 10/635,108

(22) Filed: Aug. 5, 2003

Related U.S. Application Data
(60) Provisional application No. 60/401,943, filed on Aug. 8, 2002.

(51) Int. Cl. A61K 48/00; C07H 21/02; C08G 63/91

(52) U.S. Cl. 514/44; 435/455; 525/54.2; 536/23.1

(57) ABSTRACT

This invention provides a short interfering hairpin RNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said target RNA.

This application claims the benefit of the filing date of U.S. Provisional Application No. 60/401,943, filed Aug. 8, 2002 and entitled “Short Interfering RNAs Having a Hairpin Structure Containing a Non-Nucleotide Loop,” the entire disclosure of which is hereby incorporated by reference into the present disclosure.

### BACKGROUND OF THE INVENTION

1. Field of the Invention:

The present invention relates to RNA interference, and more specifically to short hairpin interfering RNAs for gene silencing.

2. Description of the State of Art:

RNA interference RNAi has become a powerful and widely used tool for the analysis of gene function in invertebrates and plants. RNAi is the process of sequence-specific, post-transcriptional gene silencing in animals and plants, initiated by double-stranded RNA (dsRNA) that is homologous in sequence to the silenced gene. Introduction of double-stranded RNA (dsRNA) into the cells of these organisms leads to the sequence-specific destruction of endogenous RNAs that match the dsRNA. During RNAi, long dsRNA molecules are cleaved by an RNase III family nuclease call Dicer into 19- to 23-nt RNAs known as short-interfering RNAs (siRNAs). These siRNAs are incorporated into a multicomponent nuclease complex, RISC (the effector nuclease of RNAi), which identifies mRNA substrates through their homology to siRNAs and targets these cognate mRNAs for destruction. In addition, siRNAs can function as primers for an RNA-dependent RNA polymerase that synthesizes additional dsRNA, which in turn is processed into siRNAs, amplifying the effects of the original siRNAs.

Tuschl et al. first showed that short RNA duplexes, designed to mimic the products of the Dicer enzyme, could trigger RNA interference in vitro in Drosophila embryo extracts (Tuschl T., et al., Genes & Dev., 13:3191-3197 (1999)). This observation was extended to mammalian somatic cells (Elbashir, S.M. et al., Nature 411:494-498 (2001). Fire et al. have demonstrated that chemically synthesized siRNAs can induce gene silencing in a wide range of human and mouse cell lines (Nature, 391:806-811 (1998)). The use of synthetic siRNAs to transiently suppress the expression of target genes is quickly becoming a method of choice for probing gene function in mammalian cells. One limitation on siRNAs, however, is the development of continuous cell lines in which the expression of a desired target is stably silenced.

Recent studies have identified a group of small RNAs known generically as short temporal RNAs (stRNAs) and more broadly as micro-RNAs (miRNAs) in Drosophila, C. elegans, and mammals. The miRNAs appear to be transcribed as hairpin RNA precursors, which are processed to their mature, about 21 nt forms by Dicer (Lee, R. D., and Ambros, V. Science 294: 862-864 (2001)). Thus, it was realized that small, endogenously encoded hairpin RNAs could stably regulate gene expression via elements of the RNAi machinery. Neither stRNAs nor the broader group of miRNAs that has recently been discovered form perfect hairpin structures. Rather, each of these RNAs is predicted to contain several bulged nucleotides within their rather short (about 30 nt) stem structures. Only the let-7 and lin-4 miRNAs have known mRNA targets. In both cases, pairing to binding sites within the regulated transcripts is imperfect, and in the case of lin-4, the presence of a bulged nucleotide is critical to suppression (Ha, I., et al., Genes Dev. 10:3041-3050 (1996)).

Yu et al. (PNAS 99:6047-6052 (2002)) demonstrated that short hairpin siRNAs can function like siRNA duplexes to inhibit gene expression in a sequence-specific manner. Inhibition was observed both by the in vitro transcribed hairpin siRNAs by using transfection into mouse P19 cells, and by expression from the U6 promoter. The hairpin siRNAs effectively inhibited RNAs complementary to either the sense or antisense siRNA sequences.

Paddison et al. (Genes Dev. 16:948-958 (2002)) have shown that shRNAs can induce sequence-specific gene silencing in mammalian cells. It was found that silencing could be provoked by transfecting exogenously synthesized hairpins into cells, and could also be triggered by endogenous expression of shRNAs. The shRNAs were designed to include bulges within the shRNA stem, and contained nucleotide loops or varying sizes. Paddison et al. found that the stem lengths could range anywhere from 25 to 29 nucleotides and loop size could range from 4 to 23 nucleotides without affecting silencing activity.

McManus et al. (RNA 8:842-850 (2002)) also studied miRNA mimics containing 19 nucleotides of uninterrupted RNA duplex, a 12-nucleotide loop length and one asymmetric stem-loop bulge composed of a single uridine opposing a double uridine. The loop sequence was chosen from the loop of the mir-26a gene, except that a single C residue was omitted to prevent a predicted alternative nonhairpin structure. The loop was placed on the 5′ or 3′ end of the antisense strand. McManus et al. also showed that gene silencing could be provoked by transfecting exogenously synthesized hairpins into cells, and could also be triggered by endogenous expression of shRNAs.

Recently, a number of groups have developed expression vectors to continually express siRNAs in transiently and stably transfected mammalian cells (see, for example, Brummelkamp et al. (Science, 296:550-553 (2002)). Some of these vectors have been engineered to express short hairpin RNAs (shRNAs), which get processed in vivo into siRNA-like molecules capable of carrying out gene-specific silencing. The transcript folds into a stem-loop structure with 3′ UU-overhangs. The ends of the shRNAs are processed in vivo, converting the shRNAs into about 21 nucleotide siRNA-like molecules, which in turn initiate RNAi.

siRNA technology is emerging as an effective means for reducing the expression of specific gene products and may therefore prove to be uniquely useful in a number of therapeutic, diagnostic, and research applications for the modulation of expression of RIP2. However, to date the production of large amounts of shRNAs have been carried out by transfection or expression. A need therefore exists for shRNAs that can be produced in large quantities, wherein the design of the shRNA ensures that the RNA will fold back on itself to form a hairpin structure and will possess biological activity.

### SUMMARY OF THE INVENTION

Accordingly, one aspect of this invention provides a short interfering hairpin RNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said target RNA.

Another aspect of this invention provides a method for inhibiting a mRNA, comprising: a) providing an interfering hairpin RNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said target RNA; and b) contacting shRNA with a sample containing or suspected of containing the mRNA under conditions that favor intermolecular hybridization between the shRNA and the target mRNA whereby presence of the shRNA the target mRNA.

Further provided are methods of treating a plant, animal or human suspected of having or being prone to a disease or condition associated with expression of a target gene by administering a therapeutically or prophylactically effective amount of one or more of the shRNAs of the invention.

Additional advantages and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

### BRIEF DESCRIPTION OF THE FIGURES

The accompanying drawings, which are incorporated herein and form a part of the specification, illustrate preferred embodiments of the present invention, and together with the description, serve to explain the principles of the invention.

FIGS. 1A-1D illustrate non-limiting examples of short hairpin interfering RNAs of this invention, where X₁ and X₂ are nucleic acid sequences that form the double-stranded stem region and L is the non-nucleotide loop region.

FIG. 2 is an illustration of examples of C3 non-nucleotide linkers used in the shRNAs of this invention.

FIG. 3 is an illustration of examples of 9S non-nucleotide linkers used in the shRNAs of this invention.

FIG. 4 is a bar graph of the percent silencing of lamin A/C by hairpin shRNA's of this invention having right-handed loops, control duplexes, and transfection controls, where the percent inhibition is provided as the expression of Lamin A/C normalized to GAPDH in A549 cells.

FIG. 5 is a bar graph of the percent silencing of lamin A/C by hairpin shRNA's of this invention having left-handed loops, control duplexes, and transfection controls, where the percent inhibition is provided as the expression of Lamin A/C normalized to GAPDH in A549 cells.

FIG. 6 is a bar graph of the percent silencing of lamin A/C by hairpin shRNA's of this invention and transfection controls, where the percent inhibition is provided as the expression of Lamin A/C normalized to GAPDH in A549 cells.

FIG. 7 is a bar graph of the percent silencing of lamin A/C by hairpin shRNA's of this invention and transfection controls, where the percent inhibition is provided as the expression of Lamin A/C normalized to GAPDH in A549 cells.

### DETAILED DESCRIPTION OF THE PREFERRED EMBODIMENTS

This invention provides novel agents capable of mediating target-specific RNA interference or other target-specific nucleic acid mediations such as DNA methylation, More specifically, the present invention provides a short hairpin interfering RNA (shRNA) having the formula X₁—L—X₂ and capable of forming a hairpin structure, where X₁ and X₂ represent a pair of complementary or substantially complementary nucleic acid sequences and wherein at least a portion of the nucleotide sequence of either nucleic acid sequence X₁ or X₂ is complementary to a nucleotide sequence of a target mRNA to be inhibited (i.e., sense and antisense strands), and L represents a non-nucleotide linking group having sufficient length such that X₁ and X₂ form a double-stranded stem portion of the hairpin and L forms a loop. In one embodiment, X₁ and X₂ are synthetic RNA sequences, respectively, comprising between about 15 to 30 nucleotides, preferably between about 19 to about 27 nucleotides.

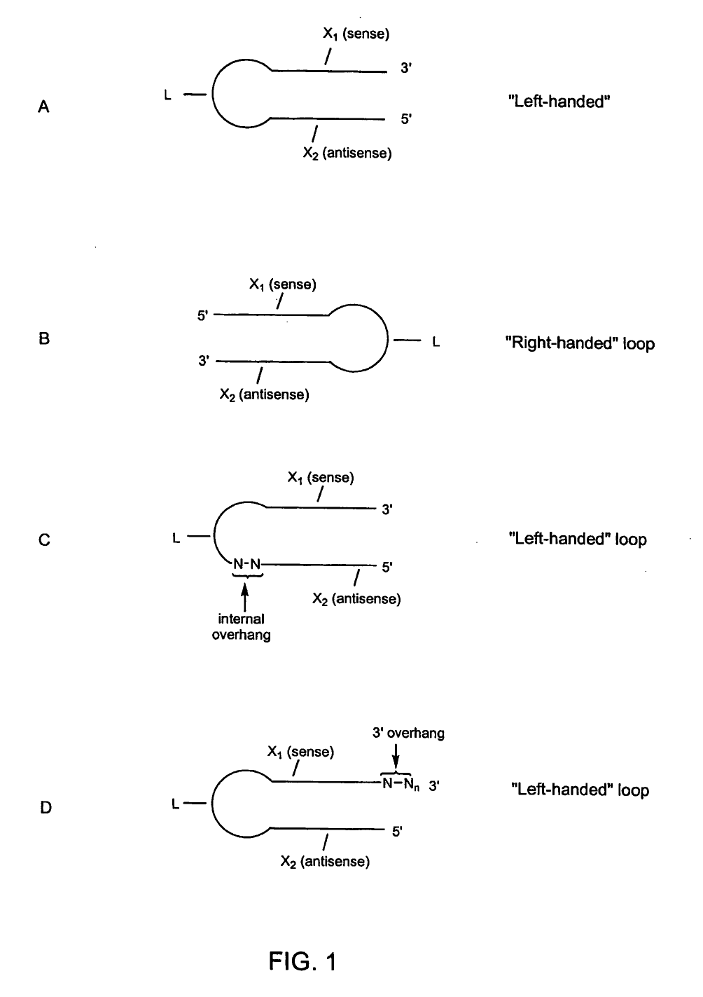

In general, certain embodiments of the shRNA of this invention can be illustrated by the non-limiting generic structures shown in FIGS. 1A-1D. It is to be understood that the examples shown in FIG. 1 are for illustration purposes only, and that many other variations of such examples are contemplated by this invention. As illustrated in FIGS. 1A-1D, “hairpin structure” refers to a structure that contains a double-stranded stem segment formed by the X₁ and X₂ sequences and a loop segment “L”, wherein the two nucleic acid strands that form the double-stranded stem segment have sufficient complementarity to one another to form a double-stranded stem hybrid and are linked and separated by a non-nucleic acid moiety that forms the loop segment. In one embodiment, the sense and antisense strands X₁ and X₂ are blunt-ended. In another embodiment, the hairpin structure comprises 3′ or 5′ single-stranded region(s) (i.e., overhangs) extending from either of the oligonucleotides X₁ or X₂ as shown in FIG. 1D. The 3′ or 5′ overhang preferably comprise between 1 and 5 nucleotides. Preferably, X₁ and X₂ are oligonucleotides that are about 19 to about 27 nucleotides in length, and X₁ exhibits complementary to X₂ of from 90 to 100%. In one embodiment, X₁ and X₂ are 100% complementary. In another embodiment, either X₁ or X₂ will further comprise a bulge or loop portion as shown in FIG. 1C and exhibit complementary of from 90 to 100% over the remainder of the oligonucleotide. The internal overhang is preferably located near or adjacent the non-nucleotide loop.

As used herein, “sequences having sufficient complementarity to one another to form a double-stranded stem hybrid” refers to a nucleic acid duplex wherein the two nucleotide strands X₁ and X₂ hybridize according to the Watson-Crick basepair principle, i.e., A-U and C-G pairs in a RNA:RNA duplex, wherein the stem may include a stem-loop bulge, e.g., composed of a single uridine opposing a double uridine.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides, such as between nucleotides in the X₁ and X₂ strands that form the stem of a shRNA molecule, or between a nucleic acid sequence of an X₁ or X₂ strand of an shRNA and a sequence in a target RNA to be inhibited. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an shRNA of this invention need not be 100% complementary to that of the target RNA to be specifically hybridizable. An shRNA is specifically hybridizable when binding of the shRNA to the target RNA molecule interferes with the normal function of the target RNA to cause a loss of utility, and there is a sufficient degree of complementarity to avoid non-specific binding of the shRNA to non-target sequences under conditions in which specific binding is desired, i.e., under physiological conditions in the case of in vivo assays or therapeutic treatment, and in the case of in vitro assays, under conditions in which the assays are performed.

The shRNAs inhibits a target RNA having a sequence complementary to either the sense (X₁) or antisense (X₂) shRNA sequences. A hairpin siRNA of this invention can target an RNA without targeting its complement equally. Further, basepairing within a hairpin shRNA duplex of this invention need not be perfect to trigger inhibition.

“Nucleotide” means either a deoxyribonucleotide or a ribonucleotide or any nucleotide analogue. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH₂, NHR, NR₂, or CN. shRNAs also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides. In one embodiment, the shRNA further comprises an element or a modification that renders the shRNA resistant to nuclease digestion.

The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Within the shRNA, at least a portion of nucleotide sequence complementary to a nucleotide sequence of a target mRNA to be inhibited must be located within the double-stranded segment. Preferably, the nucleotide sequence complementary to a nucleotide sequence of a target mRNA to be inhibited is completely located within the double stranded segment.

The oligonucleotides X₁ and X₂ that form the stem of the shRNA are separated by a flexible linker. Briefly, the flexible linker is chosen to be a non-nucleic acid moiety of sufficient length and of sufficient materials to enable effective intramolecular hybridization between oligonucleotides X₁ and X₂. The length of the linker will typically be a length which is at least the length spanned by at least 10-24 atoms, while not being so long as to interfere with either the pairing of the complementary oligonucleotides X₁ or X₂. The flexible linker can be any of a variety of chemical structures.

An shRNA can be prepared by separately synthesizing each of the oligonucleotides X₁ and X₂ and then coupling the oligonucleotides together as a single hairpin by conjugation to each end of a separately prepared flexible linker. Alternatively, a shRNA can be prepared by the phosphoramidite method described by Beaucage and Caruthers (Tetrahedron Lett., (1981) 22:1859-1862), or by the triester method according to Matteucci, et al., (J. Am. Chem. Soc., (1981) 103:3185), each of which is specifically incorporated herein by reference, or by other chemical methods using a commercial automated oligonucleotide synthesizer.

The flexible linker is provided with functional groups at each end that can be suitably protected or activated. The functional groups are covalently attached to each nucleic acid portion X₁ and X₂ via an ether, ester, carbamate, phosphate ester or amine linkage to either the 5′-hydroxyl or the 3′-hydroxyl of the probe portions chosen such that the complementary intramolecularly hybridizing sequences are in an anti-parallel configuration. When the flexible linking group L is attached to the 3′-end of the sense strand X₁ and to the 5′-end of the antisense strand X₂, the resulting loop formed by the linking group is referred to herein as a “right-handed” loop. When the flexible linking group L is attached to the 5′-end of the sense strand X₁ and to the 3′-end of the antisense strand X₂, the resulting loop formed by the linking group is referred to herein as a “left-handed” loop. Preferred linkages are phosphate ester linkages similar to typical oligonucleotide linkages. For example, hexaethyleneglycol can be protected on one terminus with a photolabile protecting group (i.e., NVOC or MeNPOC) and activated on the other terminus with 2-cyanoethyl-N,N-diisopropylamino-chlorophosphite to form a phosphoramidite. Other methods of forming ether, carbamate or amine linkages are known to those of skill in the art and particular reagents and references can be found in such texts as March, Advanced Organic Chemistry, 4th Ed., Wiley-Interscience, New York, N.Y., 1992.

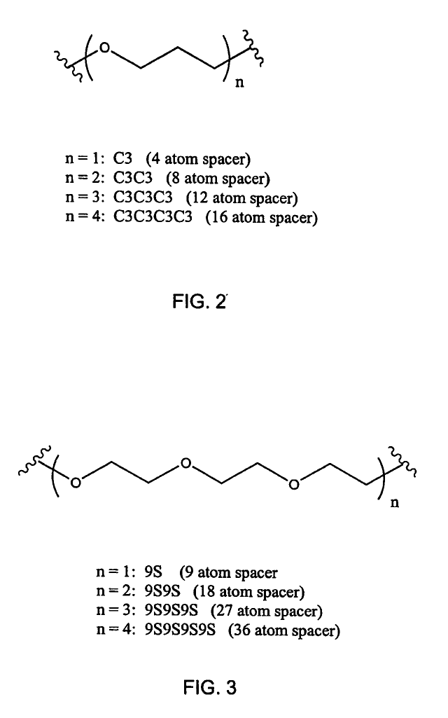

In general, the flexible linkers are non-nucleotide molecules including spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. FIGS. 2 and 3 illustrate non-limiting examples of linkers used in the shRNAs as described in Examples 1 and 2.

This invention also relates to a method for knocking down (partially or completely) a targeted gene, for example for generating models of disease states, to examine the function of a gene, to assess whether an agent acts on a gene, to validate targets for drug discovery, etc. In those instances in which gene function is eliminated, the resulting cell or organism can also be referred to as a knockout. One embodiment of the method of producing knockdown cells and organisms comprises introducing into a cell or organism (the “targeted gene”) is to be knocked down, an shRNA molecule of this invention under conditions wherein RNAi occurs, resulting in degradation of the mRNA of the targeted gene, thereby producing knockdown cells or organisms.

This invention also relates to a method for validating whether a gene product is a target for drug discovery or development. An shRNA molecule of this invention that targets the mRNA that corresponds to the gene for degradation is introduced into a cell or organism. The cell or organism is maintained under conditions in which degradation of the mRNA occurs, resulting in decreased expression of the gene. Whether decreased expression of the gene has an effect of the cell or organism is determined, wherein if decreased expression of the gene has an effect, then the gene product is a target for drug discovery or development.

The shRNAs of the present invention can be utilized for diagnostics, therapeutics, prophylaxis and as research reagents and kits. For therapeutics, a plant, animal, or human suspected of having a disease or disorder which can be treated by modulating the expression of a particular gene is treated by administering shRNA in accordance with this invention. The compounds of the invention can be utilized in pharmaceutical compositions by adding an effective amount of an shRNA to a suitable pharmaceutically acceptable diluent or carrier. Use of shRNAs and methods of the invention may also be useful prophylactically.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules for animals and humans can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀'s found to be effective in in vitro and in vivo animal models.

The shRNAs of this invention comprising non-nucleotide loops offer several advantages over shRNAs in the art comprising nucleotide loops. For example, the shRNAs of this invention are synthesized as unimolecular synthetic molecular entities, and therefore are easier to characterized and more suitable for regulatory review. In addition, synthesis of the unimolecular entities allows the introduction of non-natural entities into the shRNA (e.g., the substitution of a PEG moiety for a nucleotide), which reduces the cost of the synthesis by reducing the amount of expensive nucleotides and allow for the synthesis of shRNAs with more desirable properties (nuclease resistance, greater to support mechanisms for down regulation, increased bioavailability, increased binding to a target molecule for diagnostics, etc.). The synthesis of a unimolecular shRNA further reduces the total cost of synthesis by requiring less solid support material for the synthesis. Further, the design and structure of the shRNAs of this invention ensure that the desired hairpin structure will form during the annealing step in the absence of competing intermolecular duplexing.

### EXAMPLES

### Example 1

### Hairpin Design

The purpose of the following experiment is to test the following parameters of structure (hairpins vs. standard 21-mer duplex), length of core duplex, spacers in the loop structure, the loop being to the right or to the left, and overhang composition.

A total of 72 experimental hairpins were designed. The hairpins were designed with 6 different core sequences: 1) a 19-mer core; 2) a 21-mer core; 3) a 23-mer core; 4) a 25-mer core; 5) a 27-mer core; and 6) a 29-mer core, wherein the number of nucleotides indicates the number of nucleotides in each of the complementary sense and antisense strands, not including internal, 3′ or 5′ overhangs (see for example FIG. 1).

Each of the six different core sequences were combined with each of the following linkers, wherein the designations “C3” and “9S” are as shown in Figure X: 1) a 2-nucleotide spacer comprising cytosine and guanidine as a control; 2) a C3C3 linker (i.e, a 9 carbon linker); 3) a 9S9S linker (i.e., an 18 atom linker); 4) a C3C3C3C3 linker (i.e., an 18 carbon linker); 5) a 9S9S9S linker (i.e., a 27 atom linker); 6) a 9S9S9S9S linker (i.e., a 36 atom linker).

In addition, each of six different core sequences were linked with each of the above six linkers such that the linker formed either a as a “right” loop (“R”) or a “left” loop (“L”) in the hairpin. As used herein, a “right” loop is one that links the 3′ end of the sense strand to the 5′ end of the antisense strand, and a “left” loop is one that links the 3′ end of the antisense strand to the 5′ end of the sense strand (see Figure Y).

Finally, each hairpin had a UU internal overhang on the core strand that was connected to the non-nucleotide linker at its 3′ end, and a UdT terminal overhang at the 3′ end of the complementary strand.

In addition to the above-described 72 hairpin combinations, 7 control duplexes C1-C7 were also designed, where C3 is the linker as shown in FIG. 2 and N3 is an amino linker.

C1: a 5′-C3 linker-(19-mer sense)dTdT-3′ duplexed with a 5′-C3 linker-(19-mer antisense)dTdT-3′;

C2: a 5′-C3 linker-(19-mer sense)dTdT-3′ duplexed with a 5′-(19-mer antisense)dTdT-3′;

C3: a 5′-(19-mer sense)dTdT-3′ duplexed with a 5′-C3 linker-(19-mer antisense)dTdT-3′;

C4: a 5′-(19-mer sense)dTdT-3′ duplexed with a 5′-(19-mer antisense)dTdT-3′;

C5: a 5′-(19-mer sense)dTdT N3-3′ duplexed with a 5′-(19-mer antisense)dTdT N3-3′;

C6: a 5′-(19-mer sense)dTdT N3-3′ duplexed with a 5′-(19-mer antisense)dTdT -3′; and

C7: a 5′-(19-mer sense)dTdT -3′ duplexed with a 5′-(19-mer antisense)dTdT N3-3′.

Synthesis and Purification of Oligonucleotides:

Oligonucleotide synthesis conditions were adapted from U.S. Pat. No. 5,889,136 to Scaringe and Caruthers, which is specifically incorporated herein by reference. The hairpins and control duplexes were synthesized on a 0.2 mmol dT column using 5′-silyl-2′-ACE chemistry on a 394 ABI instrument according to the method described in U.S. Pat. No. 6,111,086 to Scaringe, which is specifically incorporated herein by reference. The protocols can be adapted by those skilled in the art to any commercially available synthesizer. Following synthesis on the synthesizer, the polymer support is treated with a IM solution of disodium 2-carbamoyl-2-cyanoethylene-1,1-dithiolate trihydrate (S₂Na₂) to remove the methyl protecting groups from the phosphates. The S₂Na₂ reagent was washed out with water and acetone. The dried support was treated with 40% N-methylamine in water at 55° C. to cleave all base-labile protecting groups and release the oligonucleotide into solution. The oligonucleotides were brought up to a volume 1.6 mL with sterile water. Two aliquots were taken for a quantity and quality assay. For quantity, a 1:100 dilution was used to read the Optical Density Units. For quality, the product was analyzed under highly denaturing conditions using polyacrylamide gel electrophoresis (PAGE) with 7M urea at 60° C. A 10 mL 2′-ACE protected RNA aliquot was electrophoresed on a 15% polyacrylamide gel. The gel was run at 40° C. for approximately 4 hours.

The oligonucleotides were 2′-deprotected under very mild acidic conditions with a tetramethylethylenediamine-acetate (TEMED-acetate) buffer, pH 3.8, for 3 minutes at 90° C. in a dry heat block. The oligonucleotides were then cooled at room temperature for 30 minutes to allow annealing (hairpin or duplex formation) and then placed in a speed-vac to dry. The duplex was resuspended in a glycerol/TBE loading buffer and electrophoresed on a 10% polyacrylamide gel. The gel was run at room temperature for 3 hours. The duplex band was visualized using UV shadowing and excised from the gel. The excised band was crushed and soaked in 0.3 M NaOAc overnight. Desalting was accomplished by filtering the duplex was filtered away from the gel using a barrel and glass wool and loading the filtered duplex onto a previously prepped and equilibrated C18 column. The column was washed with 50 mM Triethylammonium bicarbonate (TEAB) and eluted in 35% MeCN :35% MeOH:30 50 mM TEAB. An aliquot of the sample was read at A260 on a UV spectrophotometer. The duplex was then aliquoted into 3.0 optical density unit (ODU) aliquots and dried in the presence of buffer.

Transfection:

The duplexes were transfected in triplicate into A549 cells. Two negative transfection controls were used as well as a positive p53. A549 cells were plated at 20K per well in 48-well plates the day before transfection. On the day of transfection cells were about 70% confluent. Hairpins as well as two negative and one positive control were transfected at a final concentration of 50 nM complexes with 1 μg/mL of cationic lipids in growth media containing serum for 24 hours. each hairpin was transfected in triplicate. After 24 hours cells were lysed and polyA mRNA isolated using Sequitur's mRNA Catcher purification plate.

Detection:

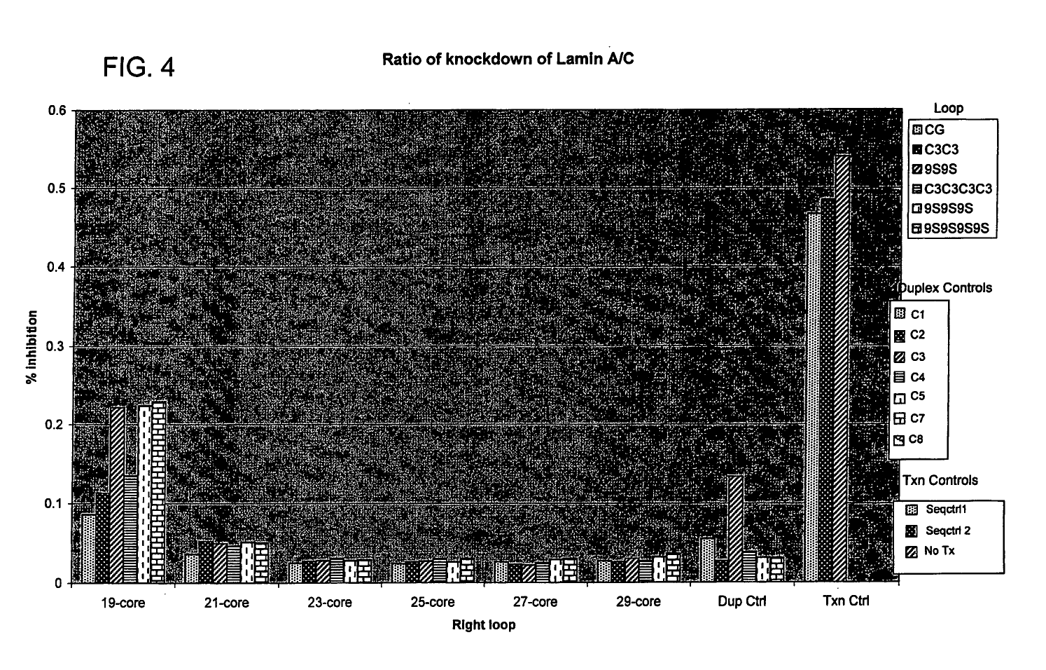

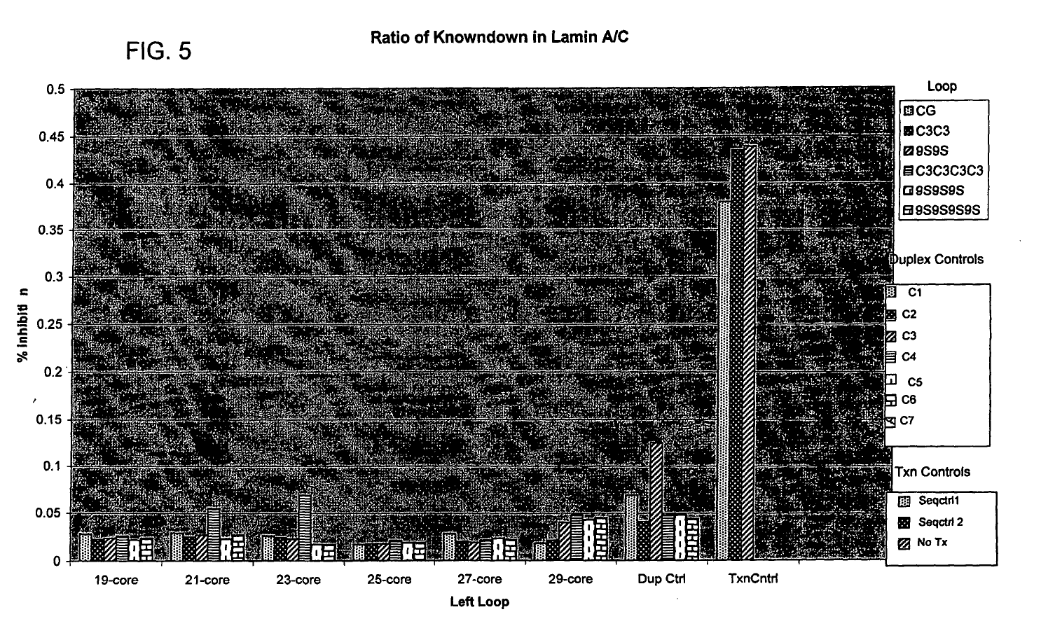

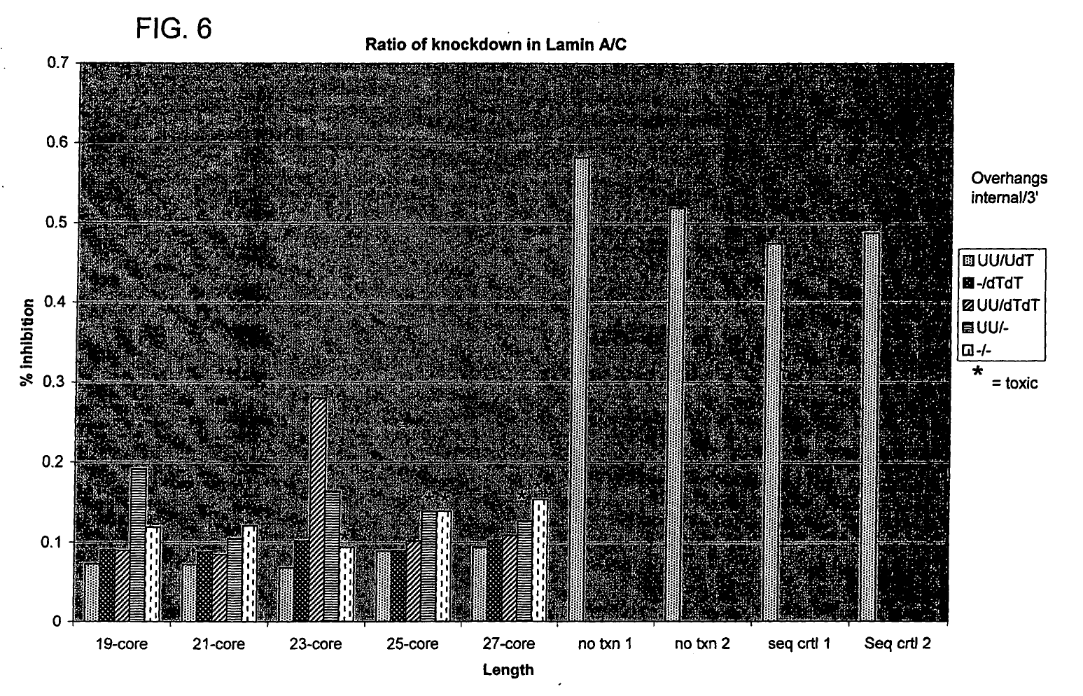

The silencing of the lamin A/C gene by RNA interference with the shRNAs or control duplexes was examined. RT-PCR analysis was performed using Sequitur's RT system, and each sample was measured in triplicate for the target gene and GAPDH. The target values were normalized to GAPDH and are shown as the average of triplicate transfections. TaqMan was used to determine the amount of knockdown for each hairpin or duplex. The results are shown in FIGS. 4 and 5, where the percent inhibition is provided as the expression of Lamin A/C normalized to GAPDH in A549 cells. FIG. 4 shows results obtained for shRNAs of this invention having right-handed loops. FIG. 5 shows the results obtained for shRNAs of this invention having left-handed loops. The percent inhibition in FIGS. 4 and 5 is provided as the expression of Lamin A/C normalized to GAPDH in A549 cells. The results shown in FIGS. 4 and 5 demonstrate that each of the core sequences, 19 through 29, with either a left or right hand loop (with the exception of the right 19-mer core) in conjunction with any spacer or nucleotide loop effectively silenced the Lamin A/C gene. However, the shRNA having left-handed loops worked more effectively then those with right-handed loops.

### Example 2

### Hairpin Design with Different Overhangs

The purpose of the following experiment was to test the parameters of overhang composition. Twenty five new experimental hairpins were designed. The hairpins were designed with one of the following 5 core sequences: 1) a 19-mer core; 2) a 21-mer core; 3) a 23-mer core; 4) a 25-mer core; and 5) a 27-mer core, wherein the number of nucleotides listed indicates the number of nucleotides in each of the sense and antisense strands, not including internal, 3′ or 5′ overhangs. All of the hairpins contained the 9S9S9S linker (see FIG. 3), and all contained “left” loops (i.e., the linker was coupled to the 5′ end of the sense strand and the 3′ end of the antisense strand). The hairpins were further designed to have one of the following overhang combinations:

| internal overhang | 3′ overhang |
|---|---|
| UU | UdT |
| — | dTdT |
| UU | dTdT |
| UU | — |
| — | — |

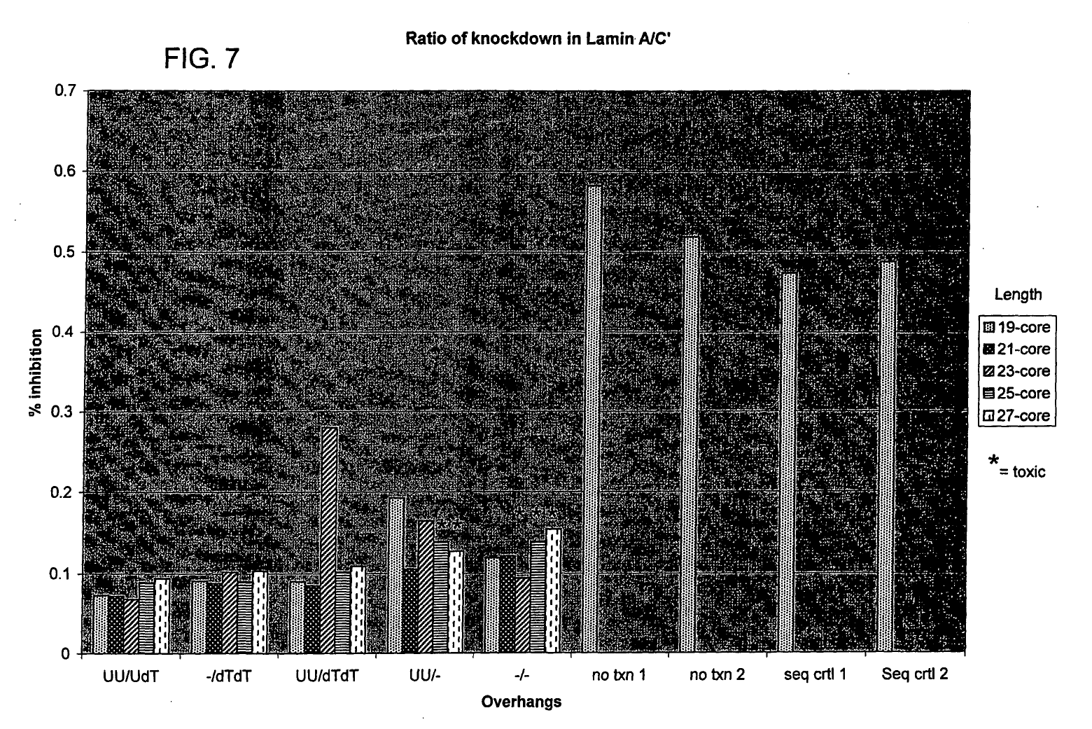

The hairpins were synthesized, annealed, purified, and transfected as described in Example 1. TaqMan® was used to determine the amount of knockdown for each duplex. The 7 control duplexes C1-C7 were also used as controls in the knockdown experiments. The silencing of the lamin A/C gene by RNA interference with the shRNAs or control duplexes were examined. The results are shown in FIGS. 6 and 7. In FIG. 6 the results are plotted by grouping hairpins of the same core length, and FIG. 7 shows the same results but grouped according to the type of overhang. The percent inhibition in FIGS. 6 and 7 is provided as the expression of Lamin A/C normalized to GAPDH in A549 cells. The results shown in FIGS. 6 and 7 demonstrate that while all of the overhang combinations at any core length were effective in silencing the Lamin A/C gene, the UU/UdT and UU/dTdT and -/dTdT were the most effective combinations. The combination of UU/- and -/- had some level of toxicity to the cells.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily occur to those skilled in the art, it is not desired to limit the invention to the exact construction and process shown described above. Accordingly, all suitable modifications and equivalents may be resorted to falling within the scope of the invention as defined by the claims that follow.

What is claimed is:

1. An interfering hairpin RNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said target RNA.

2. The hairpin RNA of claim 1, wherein each of said X₁ and X₂ nucleotide sequences comprise between about 19 to 27 nucleotides.

3. The hairpin RNA of claim 1, wherein said non-nucleotide linker L is selected from the group consisting of polyethers, polyamines, polyesters, polyphosphodiesters, alkylenes, attachments, bioconjugates, chromophores, reporter groups, dye labeled RNAs, and non-naturally occurring nucleotide analogues or combinations thereof.

4. The hairpin RNA of claim 3, wherein said polyether is selected from the group consisting of polyethylene glycol, polyalcohols, polypropylene glycol or mixtures of ethylene and propylene glycols.

5. The short interfering RNA of claim 1, wherein the double-stranded segment of the hairpin structure is formed between two perfectly matched nucleotide sequences.

6. The short interfering RNA of claim 1, wherein the double-stranded segment of the hairpin structure is formed between two imperfectly matched nucleotide sequences.

7. The short interfering RNA of claim 1, further comprising a 3′ overhang sequence.

8. The short interfering RNA of claim 1, further comprising an internal overhang.

9. A method for inhibiting a mRNA, comprising:
a) providing an interfering hairpin RNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said target RNA; and
b) contacting shRNA with a sample containing or suspected of containing the mRNA under conditions that favor intermolecular hybridization between the shRNA and the target mRNA whereby presence of the shRNA the target mRNA.

10. A method for assaying whether a gene product is a suitable target for drug discovery comprising:
a) introducing an shRNA which targets the mRNA of the gene for degradation into a cell or organism, wherein said shRNA having the structure X₁-L-X₂, wherein X₁ and X₂ are nucleotide sequences having sufficient complementarity to one another to form a double-stranded stem hybrid and L is a loop region comprising a non-nucleotide linker molecule, wherein at least a portion of one of the nucleotide sequences located within the double-stranded stem is complementary to a sequence of said double-stranded RNA;
b) maintaining the cell or organism of (a) under conditions in which degradation of the MRNA occurs, resulting in decreased expression of the gene; and
c) determining the effect of the decreased expression of the gene on the cell or organism, wherein if decreased expression has an effect, then the gene product is a target for drug discovery.
